(19) United States
(12) Patent Application Publication
(10) Pub. No.: US 2006/0058390 A1
(43) Pub. Date: Mar. 16, 2006

(54) D-methionine formulation with improved biopharmaceutical properties

(75) Inventor: Prasad Sunkara, Saline, MI (US)

Correspondence Address: STERNE, KESSLER, GOLDSTEIN & FOX PLLC, 1100 NEW YORK AVENUE, N.W., WASHINGTON, DC, 20005, US

(73) Assignee: Molecular Therapeutics, Inc., Ann Arbor, MI (US)

(21) Appl. No.: 11/225,107

(22) Filed: Sep. 14, 2005

Related U.S. Application Data
(60) Provisional application No. 60/609,255, filed on Sep. 14, 2004.

(51) Int. Cl. A61K 31/198

(52) U.S. Cl. 514/562

(57) ABSTRACT

The present invention provides pharmaceutical suspensions of D-methionine in which the aqueous solubility of D-methionine is exceeded, thereby allowing oral administration of higher doses. The present invention also provides processes for preparing these suspensions. The present invention further provides methods for preventing, treating, or ameliorating oral mucositis, hearing loss due to chemotherapy, antibiotics and noise, neuronal damage due to various CNS disorders and injuries, and anthracycline toxicity.

### CROSS REFERENCE TO RELATED APPLICATIONS

The present application claims the benefit of U.S. Provisional Patent Application No. 60/609,255, filed Sep. 14, 2004, the disclosure of which is incorporated herein by reference.

### BACKGROUND OF THE INVENTION

1. Field of the Invention

The present invention relates to an oral formulation of D-methionine comprising a suspension in which the concentration of D-methionine exceeds its aqueous solubility permitting administration of higher dosages acceptable and advantageous for oral administration to a patient.

2. Background of the Invention

Methionine is a natural micronutrient and thus is not foreign to the human body, and generally is found in the diet at a concentration of about 26 mg/g (National Research Council, 1980). D/L-methionine has historically been used as a therapeutic at relatively high doses. The World Health Organization lists D/L-methionine as an essential drug, for treating acetaminophen and paracentamol overdose and helping to regenerate the glutathione biotransformation system in the liver (WHO, 1988). As an oral antidote, D/L-methionine is often administered at 2.5 g, followed by 3 more 2.5 g doses at 4 hr intervals, for a total dose of 10 g over 12 hr. Dorfman et al. (1997) reported oral administration of 3 g L-methionine twice daily for 6 months to treat vacuolar myelopathy in 12 HIV-infected human adults and further reported that a dosage of 20 g/day for an adult is safe for chronic administration. Dorfman et al. AIDS 11:1066-1067 (1997).

L-methionine has long been used as an orally administered over-the-counter preparation to reduce urinary odor and dermatitis. For adults, the recommended dose is 200-400 mg orally 3-4 times/day. Most human studies using L-methionine reported no side-effects (Kaji et al., Res. Commun. Chem. Pathol. Pharmacol. 56:101-109 (1987); Kies et al., J. Nutr. 105:809-814 (1975); Stegink et al., J. Nutr. 116:1185-1192 (1986)). However, methionine toxicity can occur from very high doses of racemic or L-methionine, particularly in the presence of a low protein diet and/or in developing animals (Benevenga, J., Agric. Food Chem. 22:2-9 (1974)). Unlike L-methionine, D-methionine is metabolized to 2-keto-methylthiobutyrate (Blom et al., Clin. Sci. (Lond.) 76:43-49 (1989)), which is non-toxic, even at high levels (Walser et al., J. Clin. Invest. 52:2865-2877 (1973)).

D-methionine is the dextro isomer of the essential amino acid, L-methionine. Methionine acts as a free radical scavenger and synthetic substrate that can enhance the intracellular production of the key antioxidant, reduced glutathione (GSH) (Lu, 1998). D-methionine may also increase mitochondrial GSH, an effect that can prevent oxidative stress-induced apoptosis (Fernandez-Checa et al., 1998; Ghibelli et al., 1998).

D-methionine has adequate bioavailability only at high oral dosages.

The D-isomer appears to have a longer half-life and enhanced bioavailability, yet both L- and D-methionine have been reported to effectively prevent the oxidative stress-induced ototoxicity and nephrotoxicity associated with the chemotherapeutic agent, cisplatin (Campbell et al., Hearing Res. 102:90-98 (1996), Reser et al., Neurotoxicol 20:731-748 (1999)). D-methionine has been shown to prevent cisplatin induced hair cell loss in a rat model and also markedly reduce cisplatin damage to the stria vascularis (Campbell et al., Hearing Res. 138:13-28 (1999)).

In humans, D-methionine reaches higher plasma levels than L-methionine, which enhances its effectiveness as a protective agent. In humans, 60-70% of D-methionine is excreted without conversion to the L-isomer, except in L-methionine deprivation, which can increase the conversion (Benevenga, 1974; Walser et al., 1973; Stekol and Szaran, 1962; Friedman, J. Agric. Food Chem. 47:3457-3479 (1999)).

Since the solubility of D-methionine in aqueous solutions is limited (50 mg/ml), the administration of pharmacological doses of the compound in gram quantities (a total of 3-8 g per dose) to humans has been difficult. Hence, there is a need for an oral dosage form of D-methionine which provides improved bioavailability to obviate the need for frequent and multiple daily administration of gram quantities to achieve adequate exposure and blood levels for efficacy. In particular, an oral formulation that could be administered without compromising on the bioavailability would be highly advantageous for the treatment of chemotherapy induced oral mucositis. The present invention fulfills this need by providing an oral suspension formulation in which the aqueous solubility of D-methionine is greatly exceeded, thereby allowing delivery of higher doses to patients.

### SUMMARY OF THE INVENTION

In one embodiment, the present invention provides pharmaceutical suspensions, comprising: D-methionine, a suspending agent, and a solvent. The concentration of D-methionine in the suspensions of the present invention will suitably be about 20 mg/ml to about 2000 mg/ml, and more suitably about 200 mg/ml.

Suitable suspending agents for use in the practice of the present invention include, but are not limited to, poloxamers, poloxamines, polysorbates, ethoxylated monoglycerides, ethoxylated diglycerides, ethoxylated lipids, ethoxylated fatty alcohols and ethoxylated fatty acids. In certain embodiments, the suspending agent is polysorbate 80. In further embodiments, the suspensions of the present invention can comprise preservative agents, such as parabens, including methylparaben and propylparaben.

The pharmaceutical suspensions of the present invention can further comprise one more pharmaceutical excipients including preservative agents, thickening agents, humectants, sweetening agents and flavoring agents.

In one embodiment, the present invention provides a pharmaceutical suspension for oral administration, comprising: D-methionine at a concentration of about 200 mg/ml, methylparaben at a concentration of about 1 mg/ml, propylparaben at a concentration of about 0.1 mg/ml, xantan gum at a concentration of about 1.2 mg/ml, polysorbate 80 at a concentration of about 1 mg/ml, sorbitol at a concentration of about 50 mg/ml and water, and can further comprise a sweetening agent and/or a flavoring agent.

The present invention also provides processes for producing a pharmaceutical suspension, comprising: mixing D-methionine with a suspending agent and a solvent to give a suspension with a D-methionine concentration of about 20 to about 2000 mg/ml. In further embodiments, the processes of the present invention can further comprise adding one or more excipients selected from the group consisting of preservative agents, thickening agents, humectants, sweetening agents and flavoring agents.

The present invention also provides methods for preventing, treating, or ameliorating oral mucositis, hearing loss induced by chemotherapy (platinum compounds), aminoglycosides and noise, and anthracycline toxicity in a patient in need thereof comprising administering to the patient a therapeutically effective amount of D-methionine in a suspension in accordance with the present invention. The suspensions of the present invention can also be used to treat, prevent, or ameliorate neuronal damage due to CNS disorders or injuries, including neurodegenerative diseases (e.g., Alzheimer's disease, Huntington's chorea, Parkinson's disease, amyotrophic lateral sclerosis, degenerative ataxias, multiple system atrophy), cerebrovascular diseases (e.g., global or local ischemia, intracerebral hemorrhage, stroke), seizures and epilepsy, viral diseases (e.g., meningitis, encephalitis), multiple sclerosis, brain tumors, and mechanical force injury. D-methionine will suitably be at concentration of about 20 mg/ml to about 2000 mg/ml in these suspensions, and more suitably at about 200 mg/ml. In certain embodiments, the suspensions of the present invention are administered orally, once daily. When treating oral mucositis, the suspensions are suitably given during and/or after radiation and or chemotherapeutic treatments.

### BRIEF DESCRIPTION OF THE FIGURES

FIG. 1 shows the mean D-Methionine plasma concentration in male Sprague-Dawley rats following D-Methionine administration.

FIG. 2 shows the mean L-Methionine plasma concentration in male Sprague-Dawley rats following D-Methionine administration.

### DETAILED DESCRIPTION OF THE INVENTION

Suitable embodiments of the present invention are now described.

While specific configurations and arrangements are discussed, it should be understood that this is done for illustrative purposes only. A person skilled in the relevant art will recognize that other configurations and arrangements can be used without departing from the spirit and scope of the invention.

Oral Mucositis

Oral Mucositis (OM) is a painful, debilitating, and dose-limiting side-effect of radiation therapy in which the cells of the oral mucosa undergo apoptosis and ulcers are produced, often with secondary bacterial or fungal infections. These ulcers put the patient at a significantly increased risk of developing septicemia. Patients will often develop a partial (hypogeusia), or more typically a complete (ageusia), loss of taste during radiation or chemotherapeutic treatment of mouth cancers. Furthermore, cytotoxicity to the salivary glands results in xerostomia (dry mouth with discomfort, and difficulty in speech and swallowing) and can lead to bacterial decay. Pain, loss of taste, and xerostomia (the unwillingness or inability to eat) resulting from oral mucositis is a common and almost universal complaint in patients receiving external irradiation to the oral cavity. Throughout this process, patients experience weight loss, weakness, inactivity, discouragement, further anorexia, and susceptibility to infection. With the improved ability to manage other cytotoxic-related side effects (such as myelosuppression and emesis), OM has become one of the major dose-limiting side-effects of chemo and radiation therapy. (Sonis et al., J. Natl. Cancer Inst. Monogr. 29:1-2 (2001)).

Some degree of OM occurs in approximately 40% of patients who receive cancer treatment (Sonis, Oral Oncol. 33:47-54 (1997)). About one-half of those individuals develop lesions of such severity as to require modification of their cancer treatment and/or parenteral analgesia. The incidence of OM is consistently higher among patients undergoing conditioning therapy for bone marrow transplant, continuous infusion therapy for breast and colon cancer, and radiation therapy for tumors of the head and neck. Among patients in high-risk protocols, severe OM occurs with a frequency in excess of 60% (Woo et al., Cancer 72:1612-1617 (1993)). In addition to its impact on quality of life and morbidity and mortality, OM also has a significant economic cost, ranging from $20,000-30,000 per hospitalization.

The present invention provides an oral suspension of D-methionine that can be used to treat the various conditions related to OM. Similarly, this suspension can be used to treat various diseases and conditions, including, but not limited to, the treatment of radiation toxicity, the toxicity of platinum-containing anti-tumor compounds and other ototoxic drugs, the toxicity of noise, peripheral neuropathy, and the toxicity of cardiotoxic drugs such as anthracyclines (i.e., adriamycin).

In one embodiment, the present invention provides pharmaceutical suspensions, comprising: D-methionine, a suspending agent, and a solvent.

As used herein, the term suspension means a solvent in which small particles of a solid, semisolid, or liquid are uniformly dispersed, but not dissolved. Dispersion of the particles is maintained by shaking, or stirring the mixture. In the present invention, D-methionine particles are suspended using the various suspending agents noted throughout to produce a formulation with a D-methionine concentration that greatly exceeds the solubility of the compound.

The term “solvent,” as used herein, is intended to refer to aqueous solutions that may further comprise one or more co-solvents, such as alcohols (e.g., ethanol and propylene glycol), polyethylene glycols and their derivatives, glycerol and other body-tolerated solvents.

The term “D-methionine,” as used herein, is intended to refer to an optically active composition of methionine wherein the compound rotates plane polarized light clockwise (e.g., a dextrorotatory molecule) as measured by a polarimeter. Suitably, the D-methionine has an enantiomeric excess of 1% to 100%. In certain embodiments, the D-methionine has an enantiomeric excess of at least 10%, 20%, 30%, 40%, 50%, 60%, 70%, 80%, 90%, 91%, 92%, 93%, 94%, 95%, 96%, 97%, 98%, 99%, or 100%. The D-methionine can be in any form suitable for use in the present invention, including pharmaceutically acceptable salts, e.g., the chloride, iodide, dicyclohexylamine, dicyclohexylammonium, cyclohexylammonium, cyclohexylamine, sulfonate, and acetate salts.

The concentration of D-methionine that can be prepared in the suspensions of the present invention can be readily determined by one of ordinary skill in the art using standard techniques and measurements. In general, the concentration of D-methionine in the suspensions of the present invention will be from about 20 mg/ml to about 2000 mg/ml. Suitably, the concentration will be about 100 mg/ml to about 1000 mg/ml, about 100 mg/ml to about 500 mg/ml, or about 200 mg/ml.

D-methionine has a solubility of about 5% (wt/wt) or approximately 50 mg/mL in water. In accordance with the present invention, the present suspension formulations provide for at least a 4-fold increase in D-methionine per unit volume of suspension (a concentration of about 200 mg/ml). The formulations of the present invention, therefore, uniquely permit an exceptionally high dose of D-methionine to be given in the form of a suspension.

As used herein, a “suspending agent” is any agent that can be used to generate a suspension of D-methionine in a solvent system. Suitable suspending agents that can be used in the practice of the present invention include, but are not limited to sterically stabilizing substances such as poloxamers and poloxamines (block copolymers of polyoxyethylene and polyoxypropylene), ethoxylated esters of sorbitan fatty acids, including polysorbates (such as polysorbate 80 or Tween 80™), ethoxylated mono- and diglycerides, ethoxylated lipids, ethoxylated fatty alcohols, fatty acids and vitamin E-TPGS (d-alpha tocopheryl polyethylene glycol 1000 succinate). In suitable embodiments, the suspending agent will be a polysorbate, such as polysorbate 80. Appropriate concentrations of suspending agents for use in the practice of the present invention can be easily determined by those skilled in the art. The concentration of polysorbate 80 useful in preparing the suspensions of the present invention will be about 0.1 mg/ml to about 10 mg/ml, about 0.5 mg/ml to about 5 mg/ml or about 1 mg/ml.

In further embodiments, the suspensions of the present invention can further comprise preservatives. Any suitable preservatives known to those skilled in the art can be used in the suspensions of the present invention. For example, antimicrobial agents, antifungal agents, or antibacterial agents can be used. Suitable preservatives that may be used include, but are not limited to, butylated hydroxyanisole (BHA), butylated hydroxytoluene (BHT), benzyl alcohol, ethyl alcohol, parabens such as methyl-, ethyl-, propyl-, or butylparaben, chlorobutanol, sodium benzoate, benzoic acid, myristyl-gamma-picolinium chloride, benzalkonium chloride, benzethonium chloride, cetylpyridinium chloride, chlorocresol, cresol, dehydroacetic acid, methylparaben sodium, phenol, phenylethyl alcohol, potassium benzoate, potassium sorbate, propylparaben sodium, sodium dehydroacetate, sodium propionate, sorbic acid, thymol, and combinations thereof. Suitably, preservatives for use in the practice of the present invention will be parabens, such as methylparaben and propylparaben. Useful concentrations of such preservatives can routinely be determined by those skilled in the art. For example, methylparaben can be used in the suspensions of the present invention at a concentration of about 0.1 mg/ml to about 10 mg/ml, about 0.5 mg/ml to about 5 mg/ml or about 1 mg/ml, and propylparaben can be used at a concentration of about 0.01 mg/ml to about 1 mg/ml or about 0.05 mg/ml to about 0.5 mg/ml or about 0.1 mg/ml. Such preservatives can be used either alone or in combination with one another or other preservatives.

The suspensions of the present invention can also include one or more pharmaceutical excipients, such as thickening agents, humectants, sweetening agents and flavoring agents.

Examples of thickening agents include, but are not limited to, carboxypolymethylene, sodium carboxymethylcellulose, hydroxy-ethylcellulose, xantan gum, hydroxypropylmethylcellulose, methylcellulose, and carrageenan. Examples of humectants include, but are not limited to, polyhydric alcohols, polyols such as glycerol, propylene glycol, propylene glycol glycerol, polyethylene glycol, isomalt, xylitol, maltitol, sorbitol, mannitol and the like. Examples of sweetening agents include, but are not limited to, sucrose, fructose, maltose, glucose and artificial sweeteners. Examples of flavoring agents include chocolate, thalmantin, aspartame, root beer, chewing gum, watermelon, cherry, orange, mango, or other flavorings stable at pH 7 to 9.

In one embodiment, the present invention provides a pharmaceutical suspension of D-methionine as outlined in the Table 1 below.

TABLE 1

| | | |
|---|---|---|
| | Quantity in | Active/Non- |
| Ingredient | grams/60 mL | Active |

| | | | |
|---|---|---|---|
| D-Methionine | 12.000 | | ACTIVE |
| Methylparaben | 0.060 | | NON-ACTIVE |
| Propylparaben | 0.006 | | NON-ACTIVE |
| Xantan gum | 0.072 | | NON-ACTIVE |
| Polysorbate 80 | 0.060 | | NON-ACTIVE |
| (Tween-80 ™) | | | |
| Sorbitol | 3.000 | | NON-ACTIVE |
| Chewing gum, | 0.1 | mL | NON-ACTIVE |
| orange, cherry, or | | | |
| mango flavor | | | |
| Purified water | Up to 60 | mL | NON-ACTIVE |

The present invention also provides processes for preparing pharmaceutical suspensions comprising: mixing D-methionine with a suspending agent and a solvent to give D-methionine at a concentration of about 20 to about 2000 mg/ml. The processes of the present invention can further optionally comprise adding one or more excipients selected from the group consisting of preservative agents, thickening agents, humectants, sweetening agents and flavoring agents. Suitably, the concentration of D-methionine useful in the processes of the present invention will be about 200 mg/ml. The concentration of the suspending agent and the various excipients can be determined by ordinarily skilled artisans, and suitably are at concentrations as described herein.

In another embodiment, the present invention provides methods for preventing, treating, or ameliorating oral mucositis, hearing loss induced by chemotherapy (e.g., platinum compounds), aminoglycosides and noise in a patent in need thereof comprising administering to the patient a therapeutically effective amount of D-methionine in an oral suspension in accordance with the present invention. The present invention also provides methods for preventing, treating, or ameliorating anthracycline toxicity by administering a therapeutically effective amount of D-methionine in an oral suspension in accordance with the present invention.

The suspensions of the present invention can also be used to treat, prevent, or ameliorate neuronal damage due to CNS disorders or injuries, including neurodegenerative diseases (e.g., Alzheimer's disease, Huntington's chorea, Parkinson's disease, amyotrophic lateral sclerosis, degenerative ataxias, multiple system atrophy), cerebrovascular diseases (e.g., global or local ischemia, intracerebral hemorrhage, stroke), seizures and epilepsy, viral diseases (e.g., meningitis, encephalitis), multiple sclerosis, brain tumors, and mechanical force injury. D-methionine treatment of neuronal damage caused by such diseases and injuries is discussed in U.S. Provisional Patent Application No. 60/576,807, filed Jun. 4, 2004.

As used herein, the term “therapeutically effective amount” refers to that amount of the therapeutic agent sufficient to result in treatment of oral mucositis, amelioration of one or more symptoms of oral mucositis, or reduction of damage caused by oral mucositis. For example, a therapeutically effective amount preferably refers to the amount of a therapeutic agent that reduces the extent of oral mucositis by at least 10%, preferably at least 20%, at least 30%, at least 40%, at least 50%, at least 60%, at least 70%, at least 80%, at least 90%, or at least 100%. The extent of injury can be determined by any method known in the art for visualizing or measuring oral mucositis, including visual observation, blood or other bodily fluid tests, and other procedures known in the art.

The terms “prevent, preventing, and prevention,” as used herein, are intended to refer to a decrease in the occurrence of oral mucositis. The prevention may be complete, e.g., the total absence of oral mucositis. The prevention may also be partial, such that the amount of damage is less than that which would have occurred without the present invention. For example, the extent of damage using the methods of the present invention may be at least 10%, at least 20%, at least 30%, at least 40%, at least 50%, at least 60%, at least 70%, at least 80%, at least 90%, or at least 100% less than the amount of damage that would have occurred without the suspensions of the present invention.

Appropriate patient dosing, including amount and timing, using the suspensions of the present invention can readily be determined by one skilled in the art. For example, when treating oral mucositis, the suspensions of the present invention can be administered prior to, during, or after radiation treatment, or chemotherapy treatments. The duration of treatment also can be determined by the ordinarily skilled artisan, and can continue for days, weeks, months, or years, during or after treatment, as needed by the patient.

The suspensions of the present invention can be administered at a frequency sufficient to achieve a therapeutic effect, e.g., once a day, twice a day, three times a day, four times a day, etc. Suitably, the suspensions of the present invention provide a sufficient amount of D-methionine such that only a single daily dose is needed, though other dosing regimens are encompassed by the present invention. A person of skill in the art will readily understand that the exact dosing and schedule of administration of the suspensions can vary due to many factors, including, but not limited to, the age, health, weight, and past medical history of the patient, kind of concurrent treatment, frequency of treatment, route of administration, and the nature of the effect desired.

It will be readily apparent to one of ordinary skill in the relevant arts that other suitable modifications and adaptations to the methods and applications described herein can be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

Pharmacokinetics of D-Methionine

As described further below, the pharmacokinetic profile of D-methionine was investigated in male Sprague-Dawley rats. D-Methionine was given intravenously and orally in a solution formulation to rats at 150 mg/kg dose or orally in a suspension formulation in accordance with the present invention at either 150 or 300 mg/kg dose. Blood samples were collected at predetermined times and the plasma was analyzed for both D-methionine and L-methionine by an HPLC-UV assay.

Animals, Dosing and Bleeding

Twenty-six male Sprague-Dawley rats weighing approximately 200 to 300 g were used for the study. The jugular vein of six animals were cannulated and the rest of the animals were used without any further preparation.

Dosing was conducted on two occasions. On one occasion, D-methionine was administered to two groups of three rats by gavage (per os (PO)) at either 150 or 300 mg/kg dose. The drug was prepared in a suspension formulation in accordance with the present invention. The blood samples (approximately 0.5 mL/sample) were collected from each animal at predose, 15, 30 minutes, 1, 2, 4 and 6 hours postdose into microtainer tubes containing lithium heparin as an anticoagulant. Replacement blood was infused into each animal following each blood draw. On another occasion, D-methionine was administered to two groups of 10 rats one by bolus intravenous (IV) injection and one by gavage at 150 mg/kg. The drug was prepared in a solution formulation. D-methionine was dissolved in phosphate buffered saline at a concentration of 50 mg/ml. Blood samples were collected from each animal via the orbital sinus at predose, 2, 15, 30 minutes, 1, 2, 4 and 6 hours postdose. Each animal was bled twice. The blood was centrifuged immediately and the resulting plasma in tubes was flash frozen in liquid nitrogen and stored at approximately −70° C. until analysis.

Analytical Method

D-Methionine in rat plasma samples was analyzed by an HPLC-UV assay following derivatization with Marfey's reagent. L-Methinione can also be detected under the same HPLC conditions.

Pharmacokinetic Analyses

Descriptive pharmacokinetic parameters were determined by standard model independent methods (Gibaldi, M. and Perrier, D. Pharmacokinetics, Second Edition, Marcel Dekker, Inc., New York, 1982) based on the mean plasma concentration-time data. The plasma concentrations were rounded to one decimal place before the calculation. Plasma samples with concentrations below the quantifiable limit (BQL) were assigned values of zero for the calculation of mean plasma concentration.

Animal Study

The study was completed in the rats according to the sampling schedule without any complications. All animals were noted as normal and no mortality occurred during the study.

Pharmacokinetics of D-Methionine

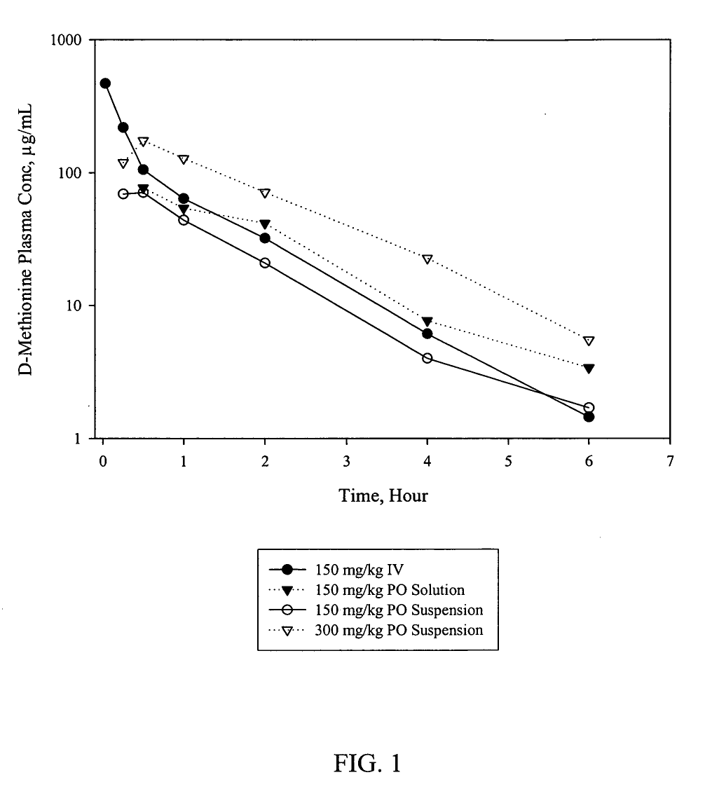

Following an intravenous dose of D-methionine at 150 mg/kg, plasma concentrations of D-Methionine declined rapidly in a multi-exponential manner with a terminal half-life was 0.9 hour (FIG. 1). The systemic clearance was 0.557 L/h/kg and the volume of distribution at steady-state was 0.50 L/kg (Table 2 below).

D-methionine was absorbed rapidly after an oral dose of 150 mg/kg from a solution formulation. Cmax of 77.10 μg/mL was observed at the first collection time point of 30 min. The area under the curve (AUC) under the concentration profile plot was 165.06 μg·h/mL which represented an absolute bioavailability of 61.3%. The terminal half-life was 1.1 hour. Similar pharmacokinetic parameters were obtained when D-methionine was given at the same dose in a suspension formulation. Increasing the dose to 300 mg/kg in the suspension formulation gave rise to a Cmax of 174.9 μg/mL and an AUC of 359.2 μg·h/mL. Absorption of D-methionine appeared to be independent of dose.

L-Methionine

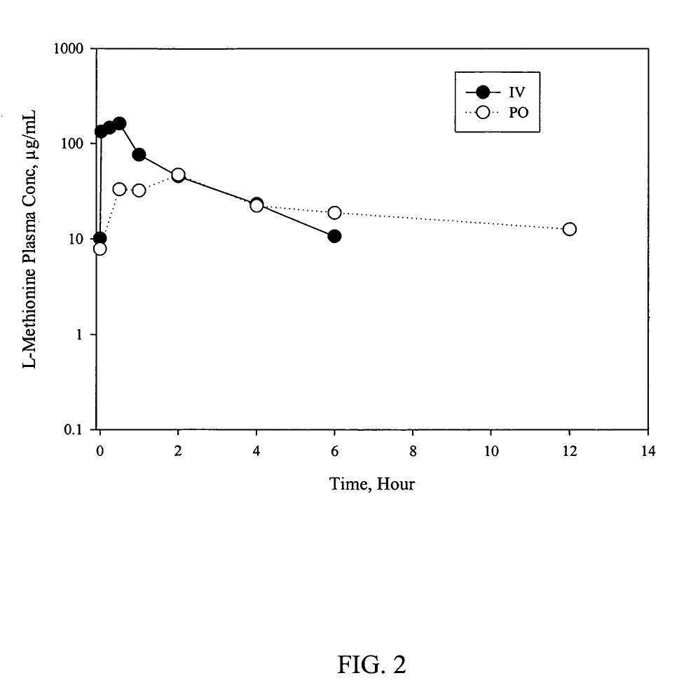

The plasma concentrations of L-methinione from the solution formulation were calculated using the calibration curve of D-methionine. The mean plasma concentration-time profiles are depicted in FIG. 2. Cmax of 161.7 and 47.2 μg/mL and AUC of 326.0 and 450.2 μg·h/mL were reached from 150 mg/kg IV and PO doses, respectively. The half-life was 1.9 and 9.9 hours for the respective route of administration (Table 3 below).

### CONCLUSION

The pharmacokinetics of D-Methionine was investigated in rats following intravenous administration at 150 mg/kg and oral administration at 150 and 300 mg/kg doses. The systemic clearance value (0.557 L/h/kg) following an intravenous administration suggests that D-methionine will not be subjected to extensive first-pass metabolism. The absolute bioavailability of 61.3% after the oral administration confirmed that D-methionine is well absorbed in the rat. The oral absorption appeared to be proportional to dose. The volume of distribution at steady-state is 0.50 L/kg and the terminal half-life is approximately one hour. L-Methionine is detected in the rat following administration in both intravenous and oral routes. The bioavailability of D-methionine was similar for both suspension and solution formulations. Thus, the suspension formulation developed helps deliver gram quantities of pharmacological doses for prevention and treatment of various indications in humans.

TABLE 2
Mean Plasma Concentrations (ng/mL) and Pharmacokinetic
Parameters of D-Methionine in Male Sprague-Dawley
Rats Following D-Methionine Administration

| | Route | | | |
|---|---|---|---|---|
| | IV | PO | PO | PO |
| | Dose, mg/kg | | | |
| | 150 | 150 | 150 | 300 |
| | Formulation | | | |
| | Solution | Solution | Suspension | Suspension |
| 0.00 h | 0.0 | 0.00 | 0.0 | 0.0 |
| 0.03 h | 470.80 | NS | NS | NS |
| 0.25 h | 219.80 | NS | 69.5 | 119.8 |
| 0.50 h | 105.95 | 77.10 | 71.1 | 174.9 |
| 1.00 h | 64.03 | 54.10 | 44.1 | 128.5 |
| 2.00 h | 32.23 | 41.67 | 20.9 | 71.4 |
| 4.00 h | 6.13 | 7.67 | 4.0 | 22.7 |
| 6.00 h | 1.45 | 3.40 | 1.7 | 5.5 |
| 12.00 h | NS | 0.00 | NS | NS |
| AUC(0-inf), μg · h/mL | 269.16 | 165.06 | 120.3 | 359.2 |
| Cmax, μg/mL | NA | 77.10 | 71.1 | 174.9 |
| Tmax, h | NA | 0.50 | 0.50 | 0.50 |
| CL, L/h/kg | 0.557 | NA | NA | NA |
| Vss, L/kg | 0.50 | NA | NA | NA |
| T_{1/2}, h | 0.9 | 1.1 | 1.0 | 1.1 |

NS = No sample
NA = Not applicable

TABLE 3
Mean Plasma Concentrations (ng/mL) and Pharmacokinetic
Parameters of L-Methionine in Male Sprague-Dawley
Rats Following D-Methionine Administration

Route

| | |
|---|---|
| IV | PO |

Dose, mg/kg

| | |
|---|---|
| 150 | 150 |

Formulation

| | Solution | Solution |
|---|---|---|
| 0.00 h | 132.6 | 7.8 |
| 0.03 h | 132.6 | NS |
| 0.25 h | 146.5 | NS |
| 0.50 h | 161.7 | 33.1 |
| 1.00 h | 76.1 | 32.0 |
| 2.00 h | 45.4 | 47.2 |
| 4.00 h | 23.2 | 22.2 |
| 6.00 h | 10.7 | 18.8 |
| 12.00 h | NS | 12.6 |
| AUC(0-inf), μg · h/mL | 326.0 | 450.2 |
| Cmax, μg/mL | 161.7 | 47.2 |
| Tmax, h | 0.50 | 2.00 |
| T_{1/2}, h | 1.9 | 9.9 |

NS = No sample

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A pharmaceutical suspension, comprising:
(a) D-methionine;
(b) a suspending agent; and
(c) a solvent.

2. The pharmaceutical suspension of claim 1, wherein said D-methionine is present at a concentration of about 20 mg/ml to about 2000 mg/ml.

3. The pharmaceutical suspension of claim 2, wherein D-methionine is present at a concentration of about 200 mg/ml.

4. The pharmaceutical suspension of claim 1, wherein said suspending agent is selected from the group consisting of poloxamers, poloxamines, polysorbates, ethoxylated monoglycerides, ethoxylated diglycerides, ethoxylated lipids, ethoxylated fatty alcohols and ethoxylated fatty acids.

5. The pharmaceutical suspension of claim 4, wherein said suspending agent is polysorbate 80.

6. The pharmaceutical suspension of claim 1, further comprising a preservative agent.

7. The pharmaceutical suspension of claim 6, wherein said preservative agent is a paraben.

8. The pharmaceutical suspension of claim 7, wherein said paraben is methylparaben and/or propylparaben.

9. The pharmaceutical suspension of claim 1, further comprising one more pharmaceutical excipients selected from the group consisting of thickening agents, humectants, sweetening agents and flavoring agents.

10. A pharmaceutical suspension for oral administration, comprising:
(a) D-methionine at a concentration of about 200 mg/ml;
(b) methylparaben at a concentration of about 1 mg/ml;
(c) propylparaben at a concentration of about 0.1 mg/ml;
(d) xantan gum at a concentration of about 1.2 mg/ml;
(e) polysorbate 80 at a concentration of about 1 mg/ml; and
(f) sorbitol at a concentration of about 50 mg/ml; and
(g) water.

11. The pharmaceutical suspension of claim 10, further comprising a sweetening agent and/or a flavoring agent.

12. A process for producing a pharmaceutical suspension, comprising:
(a) mixing D-methionine with a suspending agent and a solvent to give D-methionine at a concentration of about 20 to about 2000 mg/ml.

13. The process of claim 12, further comprising adding one or more excipients selected from the group consisting of preservative agents, thickening agents, humectants, sweetening agents and flavoring agents.

14. A method for preventing, treating, or ameliorating oral mucositis and/or hearing loss in a patient in need thereof comprising administering to the patient a therapeutically effective amount of the pharmaceutical suspension of claim 1.

15. The method of claim 14, wherein the D-methionine is at a concentration of about 20 mg/ml to about 2000 mg/ml in the suspension.

16. The method of claim 15, wherein the D-methionine is at a concentration of about 200 mg/ml in the suspension.

17. The method of claim 14, wherein the suspension is administered orally.

18. The method of claim 14, wherein the suspension is administered once a day.

19. The method of claim 14, wherein the suspension is administered more than once a day.

20. The method of claim 14, wherein the suspension is administered during and/or after radiation treatment or chemotherapeutic treatment.

21. A method for preventing, treating, or ameliorating neuronal damage due to Alzheimer's disease, Huntington's chorea, Parkinson's disease, amyotrophic lateral sclerosis, degenerative ataxias, multiple system atrophy, global or local ischemia, intracerebral hemorrhage, stroke, seizures, epilepsy, meningitis, encephalitis multiple sclerosis, brain tumors, or mechanical force injury in a patient in need thereof comprising administering to the patient a therapeutically effective amount of the pharmaceutical suspension of claim 1.

22. The method of claim 21, wherein the D-methionine is at a concentration of about 20 mg/ml to about 2000 mg/ml in the suspension.

23. The method of claim 22, wherein the D-methionine is at a concentration of about 200 mg/ml in the suspension.

24. The method of claim 21, wherein the suspension is administered orally.

25. The method of claim 21, wherein the suspension is administered once a day.

26. The method of claim 21, wherein the suspension is administered more than once a day.

27. A method for preventing, treating, or ameliorating anthracycline toxicity in a patient in need thereof comprising administering to the patient a therapeutically effective amount of the pharmaceutical suspension of claim 1.

28. The method of claim 27, wherein the D-methionine is at a concentration of about 20 mg/ml to about 2000 mg/ml in the suspension.

29. The method of claim 28, wherein the D-methionine is at a concentration of about 200 mg/ml in the suspension.

30. The method of claim 27, wherein the suspension is administered orally.

31. The method of claim 27, wherein the suspension is administered once a day.

32. The method of claim 27, wherein the suspension is administered more than once a day.
